(19) United States
(12) Patent Application Publication
(10) Pub. No.: US 2013/0158385 A1
(43) Pub. Date: Jun. 20, 2013

(54) Therapeutic Ultrasound for Use with Magnetic Resonance

(71) Applicants: Stephen R. Barnes, Bellevue, WA (US); Jerry Hopple, Seattle, WA (US); John Kook, Seattle, WA (US); Thomas R. Clary, Sammamish, WA (US)

(72) Inventors: Stephen R. Barnes, Bellevue, WA (US); Jerry Hopple, Seattle, WA (US); John Kook, Seattle, WA (US); Thomas R. Clary, Sammamish, WA (US)

(73) Assignees: Siemens Medical Solutions USA, Inc., Malvern, PA (US); Siemens Corporation, Iselin, NJ (US)

(21) Appl. No.: 13/463,693

(22) Filed: May 3, 2012

Related U.S. Application Data
(60) Provisional application No. 61/576,926, filed on Dec. 16, 2011.

(51) Int. Cl. A61N 7/00; A61B 5/055

(52) U.S. Cl. 600/411

(57) ABSTRACT

Therapeutic ultrasound applicator is provided for use with magnetic resonance system. An array of many elements, such as a multi-dimensional array, is used. To avoid cabling, the transmitters are positioned at the array. The array and transmitters are shielded to reduce interference. To avoid large inductors for the many elements, an acoustic matching layer may be sized to provide a desired phase angle or electrical impedance matching.

### RELATED APPLICATIONS

The present patent document claims the benefit of the filing date under 35 U.S.C. §119(e) of Provisional U.S. Patent Application Ser. No. 61/576,926, filed Dec. 16, 2011, which is hereby incorporated by reference.

### BACKGROUND

The present embodiments relate to therapeutic ultrasound. In particular, therapeutic ultrasound is provided for use with magnetic resonance imaging.

System design of magnetic resonance (MR) compatible equipment requires careful attention to the magnetic properties of components. Electromagnetic interference between the active circuitry and the radio frequency (RF) coils of the MR system is to be avoided. To minimize interference, only the necessary ultrasound components are placed within the bore of the MR machine. For example, the transducer array is positioned in the bore. Cabling (e.g. coaxial cables) connects the rest of the equipment (e.g., transmitters) to the transducer. The transmitters or drivers may be outside the room or Faraday cage surrounding the MR system. By placing the drive electronics and the controlling intelligence outside the MR bore, their interference with the MR magnetic fields and RF signal pickup are minimized. However, one coax cable is needed for each element of the transducer. The cabling itself is a potential avenue of interference and is normally heavily shielded to prevent both emission and susceptibility issues. Thus, the number of elements may be limited, such as 128 or 256 elements. The physical separation between the drive amplifiers and the transducer means that there is a power transfer efficiency compromise, since the coax cabling passes both forward and reflected power.

To reduce the cabling, a single spherical element may be used, requiring only one coax cable. To steer the acoustic energy, the element is mechanically moved. However, typical magnetic based motors and metal translation stages for moving the element may distort the main magnetic field of the MR system.

In one approach, about 256 elements are arranged as a tightly packed ensemble in a spherical bowl. Element phase control allows electronic beam steering over a limited angle, such as about 7°. Transmitter phasing electronics are remotely located via a large coax cable bundle. Further steering is provided with translation in three axes and rotation about two axes. The robot and array are built into an MR exam table.

### BRIEF SUMMARY

By way of introduction, the preferred embodiments described below include methods, systems, instructions, and computer readable media for therapeutic ultrasound in use with magnetic resonance. An array of many elements, such as a multi-dimensional array, is used. To avoid cabling, the transmitters are positioned at the array. The array and transmitters are shielded to reduce interference. To avoid large inductors for the many elements, an acoustic matching layer may be sized to provide a desired phase angle or electrical impedance matching.

In a first aspect, a system is provided for therapeutic ultrasound in use with magnetic resonance. A transducer array includes a multi-dimensional array of elements. A transmit beamformer connects with the transducer array. A communications interface connects with the transmit beamformer. A housing electromagnetically shields and encloses the transducer array, the transmit beamformer and the communications interface. The transducer array, the transmit beamformer and communications interface are operable in a bore of a magnetic resonance imaging system.

In a second aspect, a method is provided for therapeutic ultrasound in use with magnetic resonance. An acoustic array of elements distributed multi-dimensionally is positioned within a bore of a magnetic resonance system. The elements are driven with transmitters within the bore. Therapeutic ultrasound is applied to a patient within the bore in response to the driving. The patient is imaged with the magnetic resonance system.

In a third aspect, an ultrasound transducer includes an acoustic array of elements. A transmit beamformer has channels connected with the elements, respectively. A matching layer is adjacent to an emitting face of the acoustic array. The thickness of the matching layer off-sets a capacitance of the elements such that a phase angle of electrical impedance is within about 10 degrees of zero. The connections between the transmit beamformer and the elements is free of any matching inductors.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for therapeutic ultrasound in use with magnetic resonance;

FIG. 2 is a cross-sectional illustration of one embodiment of a combined therapeutic ultrasound and MR imaging system;

FIG. 3 is a cross-sectional diagram of one embodiment of a system for therapeutic ultrasound in use with magnetic resonance;

FIG. 4 is a block diagram of an integrated transmit beamformer and transducers according to one embodiment;

FIG. 5 is an example module for a therapeutic ultrasound applicator;

FIG. 6 is an example arrangement for therapeutic ultrasound in use with magnetic resonance; and

FIG. 7 is a flow chart diagram of one embodiment of a method for therapeutic ultrasound in use with magnetic resonance.

### DETAILED DESCRIPTION OF THE DRAWINGS AND PRESENTLY PREFERRED EMBODIMENTS

A compact, highly integrated, high multi-dimensional element count high power focused ultrasound (FUS) system is contained within a patient applicator. The multi-dimensional FUS system, with collocated drive electronics and computational resources, results in a simplified MR compatible system. The FUS system may be used in other applications than MR, such as for ultrasound imaging or therapy. Integration of the system control, beamforming computation and high power transmitters within the patient applicator allows for a large number of array elements. Cabling for the different elements is not needed, reducing interference potential when used with MR imaging. The ultrasound beam may be electronically steered over a large region, eliminating the high conductor count cable, the large cabinet of electronics, and robotic positioning of the array. The need for magnetic electrical matching components for each channel may be eliminated.

Two electrical connections, DC power and a low bandwidth communication link, may be used. The patient applicator uses DC power, cool pumped fluid, and/or a communication link to pass high level descriptive commands regarding the therapeutic ultrasound energy deposition, such as transmit frequency, power intensity, duration, duty cycle, and focus location. An enclosure and minimal cabling provide electromagnetic interference (EMI) shielding. There is no requirement for a bundle of coax cables (or other controlled impedance interconnect) or high bandwidth digital data link. The result is a compact, efficient multi-dimensional system, capable of operation from only one single-phase 3 KVA electrical circuit. Given the nature of the control signals, the supporting cart or cabinet with user interface may be positioned outside of the Faraday cage or MR room.

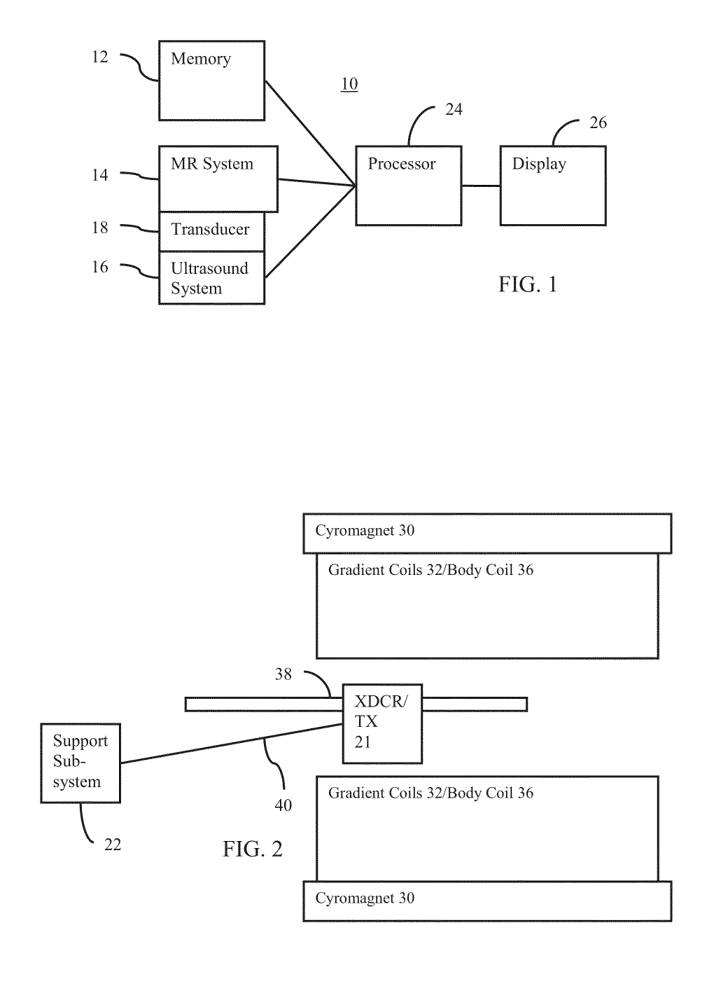

FIG. 1 shows a system 10 for therapeutic ultrasound in use with magnetic resonance. The system 10 includes a memory 12, an MR system 14, an ultrasound system 16, a transducer 18, a processor 24, and a display 26. The ultrasound system 16 and the transducer 18 are for use with the MR system. The ultrasound system 16 and the transducer 18 may be sub-divided into a sub-system 22 and an applicator 21, as shown in FIG. 2. FIG. 2 shows one example of positioning of the transducer 18 and the ultrasound system 16 relative to the MR system components.

Additional, different, or fewer components may be provided. For example, a network or network connection is provided, such as for networking with a medical imaging network or data archival system. As another example, the MR system 14, the processor 24, the memory 12, and/or the display 26 are not provided.

The memory 12, processor 24 and display 26 are part of a medical imaging system, such as the therapy ultrasound system 16, MR system 14, or other system. Alternatively, the memory 12, processor 24 and display 26 are part of an archival and/or image processing system, such as associated with a medical records database workstation or server. In other embodiments, the memory 12, processor 24 and display 26 are a personal computer, such as desktop or laptop, a workstation, a server, a network, or combinations thereof.

The display 26 is a monitor, LCD, projector, plasma display, CRT, printer, or other now known or later developed devise for outputting visual information. The display 26 receives images, graphics, or other information from the processor 24, memory 12, MR system 14, or ultrasound system 16. Where the ultrasound system 16 is for therapy only, the display 26 may be used for MR imaging and a user interface to guide steering of the therapy without displaying ultrasound images.

In one embodiment, the MR system 14 is used to generate one or more images representing tissues of a patient for display on the display 26. For example, an image or images rendered from a three-dimensional data set of MR anatomy information is provided. A multi-planar reconstruction may be provided. The user may indicate a location for treatment of the patient on the image. Alternatively, the processor 24 identifies the location for treatment.

The ultrasound system 16 is any now known or later developed ultrasound therapy system. For example, the ultrasound system 16 includes the transducer 18 for converting between acoustic and electrical energies. A transmit beamformer relatively delays and apodizes signals for different elements of the transducer 18. In alternative embodiments, the ultrasound system 16 includes a receive beamformer for generating ultrasound images. The ultrasound images may be used in addition to or as an alternative to MR imaging for therapy guidance.

Referring to FIG. 2, the magnetic resonance (MR) system 14 includes a cyromagnet 30, gradient coil 32, and body coil 36 in an RF cabin, such as a room isolated by a Faraday cage. A tubular or laterally open examination subject bore encloses a field of view. A more open arrangement may be provided. A patient bed 38 (e.g., a patient gurney or table) supports an examination subject, such as a patient with or without one or more local coils. The patient bed 38 may be moved into the examination subject bore in order to generate images of the patient. Received signals may be transmitted by the local coil arrangement to the MR receiver via, for example, coaxial cable or radio link (e.g., via antennas) for localization.

Other parts of the MR system 14 are provided within a same housing, within a same room (e.g., within the radio frequency cabin), within a same facility, or connected remotely. The other parts of the MR system 14 may include local coils, cooling systems, pulse generation systems, image processing systems, and user interface systems. Any now known or later developed MR imaging system 14 may be used. The location of the different components of the MR system is within or outside the RF cabin, such as the image processing, tomography, power generation, and user interface components being outside the RF cabin. Power cables, cooling lines, and communication cables connect the pulse generation, magnet control, and detection systems within the RF cabin with the components outside the RF cabin through a filter plate.

The MR system 14 is configured by software, hardware, or both to acquire data representing a plane or volume in the patient. In order to examine the patient, different magnetic fields are temporally and spatially coordinated with one another for application to the patient. The cyromagnet 30 generates a strong static main magnetic field B₀ in the range of, for example, 0.2 Tesla to 3 Tesla or more. The main magnetic field B₀ is approximately homogeneous in the field of view.

The nuclear spins of atomic nuclei of the patient are excited via magnetic radio-frequency excitation pulses that are transmitted via a radio-frequency antenna, such as a whole body coil 36 and/or a local coil. Radio-frequency excitation pulses are generated, for example, by a pulse generation unit controlled by a pulse sequence control unit. After being amplified using a radio-frequency amplifier, the radio-frequency excitation pulses are routed to the body coil 36 and/or local coils. The body coil 36 is a single-part or includes multiple coils. The signals are at a given frequency band. For example, the MR frequency for a 3 Tesla system is about 123 MHz+/−500 KHz. Different center frequencies and/or bandwidths may be used.

The gradient coils 32 radiate magnetic gradient fields in the course of a measurement in order to produce selective layer excitation and for spatial encoding of the measurement signal. The gradient coils 32 are controlled by a gradient coil control unit that, like the pulse generation unit, is connected to the pulse sequence control unit.

The signals emitted by the excited nuclear spins are received by the local coil and/or body coil 36. In some MR tomography procedures, images having a high signal-to-noise ratio (SNR) may be recorded using local coil arrangements (e.g., loops, local coils). The local coil arrangements (e.g., antenna systems) are disposed in the immediate vicinity of the examination subject on (anterior), under (posterior), or in the patient. The received signals are amplified by associated radio-frequency preamplifiers, transmitted in analog or digitized form, and processed further and digitized by the MR receiver.

The recorded measured data is stored in digitized form as complex numeric values in a k-space matrix. A one or multidimensional Fourier transform reconstructs the object or patient space from the k-space matrix data.

The MR system 14 may be configured to acquire different types of data. For example, the MR data represents the anatomy of the patient. The MR data represents the response to the magnetic fields and radio-frequency pulses of tissue. Any tissue may be represented, such as soft tissue, bone, or blood. The MR system 14 may be configured for acquiring specialized functional or anatomic information. For example, T1-weighted, diffusion, thermometry, or T2-weighted MR data is acquired. The MR system 14 may be configured for acquiring elastography information. Any MR elastography scan may be used. The transducer 18 may be used to induce a mechanical wave within the patient for MR imaging of strain or elasticity.

In another embodiment, the MR system 14 is not provided. The transducer 18 and ultrasound system 16 may be used outside of the MR context.

The memory 12 is a graphics processing memory, a video random access memory, a random access memory, system memory, random access memory, cache memory, hard drive, optical media, magnetic media, flash drive, buffer, database, combinations thereof, or other now known or later developed memory device for storing data or video information. The memory 12 is part of an imaging system, part of a computer associated with the processor 24, part of a database, part of another system, or a standalone device.

The memory 12 stores one or more datasets representing a three-dimensional patient volume or a two-dimensional patient plane. The patient volume or plane is a region of the patient, such as a region within the chest, abdomen, leg, head, arm, or combinations thereof. The patient volume is a region scanned by the MR system 14.

Any type of data may be stored, such as medical image data. The data represents the patient prior to or during treatment or other procedure. For example, MR anatomy data is acquired prior to a procedure, such as just prior to (minutes or seconds) or during a previous appointment on a different day. This stored data represents tissue, preferably in a high resolution.

For volume data, the stored data is interpolated or converted to an evenly spaced three-dimensional grid or is in a scan format. Each datum is associated with a different volume location (voxel) in the patient volume. Each volume location is the same size and shape within the dataset. Volume locations with different sizes, shapes, or numbers along a dimension may be included in a same dataset. The voxel size and/or distribution may be different for different types of MR data.

The memory 12 may include calibration, fiducial, or transform data relating the coordinates of the transducer 18 and ultrasound system 16 to the MR system 14. The data coordinate system represents the position of the scanning device relative to the patient, so is the same or may be directly transformed between the MR system 14 and the ultrasound system 16. For example, fiducials that a detectable in MR data are positioned on or at a known position relative to the transducer 18. A transform is generated from the fiducials to relate the two coordinate systems. The MR data may be used to directly detect the transducer 18 for generating the transform. In another alternative, the position of the transducer 18 is fixed relative to the MR system 14, so a predetermined transform may be used. In yet another alternative embodiment, the effects (e.g., temperature or tissue displacement) of ultrasound transmissions are detected with MR scanning and used to relate the coordinates.

The memory 12 or other memory is a non-transitory computer readable storage medium storing data representing instructions executable by the programmed processor 24 for ultrasound therapy in an MR environment. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 24 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for guiding therapy, registering, and/or generating images. The processor 24 is a single device or multiple devices operating in serial, parallel, or separately. The processor 24 may be a main processor of a computer, such as a laptop or desktop computer, or may be a processor for handling tasks in a larger system, such as in an imaging system (e.g., the MR system 14). The processor 24 is configured by software and/or hardware.

The processor 24 is configured to calculate a transform relating coordinate systems. The processor 24 may be configured to generate MR images. In one embodiment, the processor 24 controls interaction between the MR system 14, the user, and the ultrasound system 16. The location for treatment and/or treatment parameters (e.g., frequency, duration, duty cycle, waveform, aperture, and/or amplitude) are determined by the processor 24. Corresponding control signals are provided to the ultrasound system 16. The processor 24 may generate the user interface for control of the ultrasound system 16.

The processor 24 may be configured to trigger operation of the transducer 18 and/or the MR system 14. The scanning and therapy may be interleaved. The scanning and therapy may be caused to occur at a same time.

The processor 24 may be configured to control a mode of operation of the ultrasound system 16. For therapy, modes of operation may include testing operation (e.g., generating a sample therapeutic beam), application of therapy, calibration, displacement generation for transform determination, or other modes.

The ultrasound system 16 and the transducer 18 are adapted for use in the MR environment. Despite the MR system 14 being susceptible to even very small electromagnetic interference, more than the transducer 18 is positioned in the bore and corresponding main magnetic field. At least some active electronics or circuits are provided with the transducer 18. For example, the transmit beamformer and a communications interface are provided with the transducer 18 in an applicator 21. Providing the transmit beamformer at the transducer 18 avoids electrical impedance concerns associated with long cabling. An array of many elements may be provided since a corresponding many coaxial cables are not needed, avoiding electromagnetic interference associated with the coaxial cables.

As represented in FIG. 2, a portion or sub-system 22 of the ultrasound system 16 is spaced from the MR system 14 or at least the bore and main magnetic field. The sub-system 22 provides for user interface and high level or general control functions of therapeutic ultrasound. For example, the processor 24 is part of the sub-system 22. These control and user interface functions may be integrated into the MR system 14 or therapy system 16.

The connection 40 between the transducer 18 and the sub-system 22 may be one, two, or few number of cables. For example, the connection 40 is an optical cable or fiber optic cable for transmitting control signals to the transducer 18. Separate connections may be provided for trigger and/or mode selection, or the same cable is used. The connection 40 may include a pipe, tube, or hose for fluid.

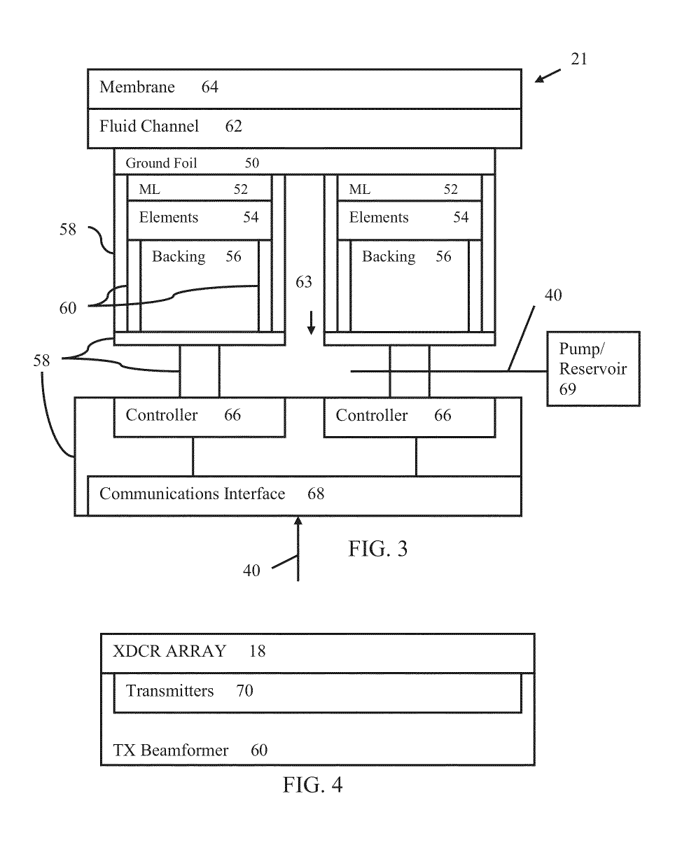

FIG. 3 shows one example embodiment of a compact, integrated therapeutic ultrasound system for use, at least in part, with the MR system 14. The system includes the elements 54 of the transducer array 18, a ground foil 50 for the elements 54, an acoustic matching layer 52, acoustically absorbing backing 56, a housing 58, a transmit beamformer 60, fluid channel 62, fluid channel 63, membrane 64, controllers 66, and a communications interface 68. Additional, different, or fewer components may be provided. For example, the fluid channels 62, 63 and membrane 64 are not provided. As another example, the communications interface 68, controller 66, and/or transmit beamformer 60 are combined together, such as on a semiconductor.

The elements 54, backing 56, matching layer 52, and/or ground foil 50 may be considered part of the transducer 18 for converting between electrical and acoustical energy. The transducer 18 may include additional components, such as a signal electrode for each element 54.

The housing 58 limits or prevents electromagnetic interference. The housing 58 electromagnetically shields and encloses the transducer array 18, the transmit beamformer 60 and the communications interface 68. The transducer 18 and drive electronics are a fully contained unit, with computational resources or precomputed parameter tables and the drive amplifiers all inside an EMI shielded enclosure of the housing 58. System transmitters 70, beamformer 60, communications interface 68, and high element count array 18 are located in close proximity to the patient and each other.

For therapeutic ultrasound, the housing 58 does not enclose a receive beamformer. A receive beamformer is not provided as part of the ultrasound system 16. Alternatively, a receive beamformer is provided, such as on a same application specific integrated circuit as the transmit beamformer 60 or on a separate component adjacent to the transmit beamformer 60.

The housing 58 has any shape. In the embodiment shown in FIG. 3, the housing 58 extends around the transducer 18 and the transmit beamformer 60. The housing 58 includes a neck region connecting between the transducer 18 and the controller 66. The controller 66 and communications interface 68 are within the same housing 58. The housing 58 may include compartments or separation of components to limit electromagnetic interference. For example, the communications interface 68 is on a printed circuit board. The housing 58 surrounds the printed circuit board other than a gap for input and/or output wires, traces, or cables (e.g., flexible circuit input/output). Similarly, the controller 66 is in a separate chamber of the housing 58 other than a gap for input and/or output wires, traces, or cables, such as serial data and power for the transmit beamformer 60. Other housing arrangements may be provided, such as positioning the controller 66 and/or the communications interface 68 within a same box or chamber as the transmit beamformer 60 with the transducer 18 in the same or different housing or chamber. Separate housings 58 may be provided for the different components.

In one embodiment, the housing 58 is a brass or copper box or cube, at least for the transducer portion. For example, the housing 58 includes four lateral sides with an open top and bottom for manufacture of the transducer 18. The top of the box is formed from the ground foil 50. The matching layer 52, the elements 54, the backing 56, and the transmit beamformer 60 are within this chamber or box of the housing 58.

The ground foil 50 is copper, aluminum or other conductive foil. An adhesive, such as silicone, epoxy, or solder, seals the ground foil 50 to the housing 58. The adhesive includes conductive particles or the ground foil 50 is in contact with the conductive housing 58 for grounding.

For manufacture, the back of the box between the backing 56 and the controller 66 is a plate, such as a plate of copper or brass. After insertion or forming of the transducer 18 in the housing 58, the back plate is connected with and sealed to the side walls of the housing 58. A gap may be provided in the back plate for flexible circuit material. The flexible circuit material is used for routing traces to electrically connect the transmit beamformer 60 to the controller 66.

The housing 58 is sealed such that fluids may not enter. For example, the use of adhesive silicone, epoxy, or solder may both hold parts of the housing 58 together and also provide a water tight seal. In alternative embodiments, a fluid tight seal is not used.

In one embodiment, a single housing 58 is used for a given transducer 18. In the embodiment shown in FIG. 3, a modular approach is used. The transducer 18, transmit beamformer 60, and controller 66 are provided in each module. Each module corresponds to a sub-array, such as a 40×40 arrangement of elements 54. To form the overall transducer 18, a plurality of modules is positioned adjacent to each other. Each module includes a separate housing 58, but a common housing 58 may be used. The transducer array 18 is constructed of self-contained sub-arrays which include sub-arrays of elements 54 and transmit beamformers 60.

Any arrangement may be provided within a given module. In the embodiment shown in FIG. 3, the semiconductor chip or chips forming the transmit beamformer 60 are thermally bonded to the housing 58. On an opposite side of the chip from the housing, flexible circuit material connects input and output pads with the elements 54 and the controller 66. The ground foil 50 seals the transducer 18 within the housing 58 of the module. While the elements 54 are shown extending to the housing 58, the elements 54 may have less lateral extent. Similarly, the transmit beamformer 60 may have less height, allowing the backing 56 to be positioned behind all of the elements 54 of the module. The elements 54 are positioned against the ground foil for transduction. One or more acoustic matching layers 52 may be between the elements 54 and the ground foil 50. Alternatively, the matching layer 52 may be outside the module on the other side of the ground foil 50.

The modules are positioned in a flat plane to form a flat emitting face of the transducer 18. Alternatively, the modules are positioned to form a curved surface for focusing and/or conforming to the patient. The connection between the modules may be flexible. Alternatively, the connection is rigid, such as bonding the modules to the housing to a flat or curved upper plate of the housing 58 of the communications interface 68. Similarly, the elements 54 in each module are arranged over a flat or curved surface with or without an ability to flex relative to each other.

The housing 58, whether for a module, group of modules, or the overall transducer 18 and transmit beamformer 60, may be sized, shaped, or arranged to connect with the patient table 38 of the MR system 14. For example, using four, sixteen, or other numbers of the modules, the applicator 21 for therapeutic ultrasound is about 2-3 inches thick (i.e., height) and about 6×8 inches on the sides. The applicator occupies less than 0.2 cubic meter. Other smaller or larger volumes may be provided. A separate housing may surround or form an outer enclosure for the applicator 21. Alternatively, at least part of the applicator's outer housing is formed by the module housing 58 and/or membrane 64.

This applicator 21 is positioned in an indention or hole in the table 38. The applicator 21 may be raised relative to the table to allow contact with the patient. Inflatable chambers and/or other robotic devices may be used to move the applicator 21 into and out of contact with the patient lying on the patient table 38. In alternative embodiments, the applicator 21 is for handheld use, part of a cuff or blanket to be worn by the patient, or positioned on an arm or other device for setting the applicator 21 adjacent to the patient while in the bore of the MR system 14. In yet other embodiments, the applicator 21 is thin enough to lay on top of the table without alteration of the table. For example, the application 21 has a thickness similar to cushions on the table.

Whether formed as a single array or as a collection of sub-arrays, the transducer 18 includes a plurality of elements 54. The transducer 18 is a multi-dimensional array of piezoelectric or capacitive membrane elements. The elements are distributed along a rectangular, triangular or other grid pattern over two dimensions, such as N×M elements where both N and M are greater than 1.

For modules, the elements 54 of the array may include gaps. The gaps may be about one to ten elements wide. Since the elements 54 of the different modules are used as part of the same aperture for therapeutic transmission, the elements 54 from the different modules are part of the same transducer array 18.

Any number of elements 54 may be used. In one embodiment, there are at least 1,600 elements. An efficient, high power, high channel count high intensity ultrasound array system may have more than 1,500 elements for providing up to 3.3 KVA power for system and all support functions and capable of producing greater than 150 acoustic watts of applied acoustic energy. Using sixteen modules of 40×40 arrangements of elements 54 may allow for over 25,000 elements in one array. In one embodiment, sixteen modules of 1,152 elements each are arranged in a 2×8 arrangement for around 16,000 elements.

The transmit beamformer 60 is an application specific integrated circuit. Discrete components, processors, field programmable gate arrays, memories, digital-to-analog converters, or other devices may alternatively or additionally be used. For a given sub-array or for the entire array 18, one or more transmit beamformers 60 may be used. For example, two, three, or four separate chips are provided for a 40×40 or other sub-array. In one embodiment, each module has 12×36 elements with 32 transmitter chips (36 channels each) and 16 beamformer chips (72 channels each)). 228 channels or other numbers per chip may be used.

The transmit beamformer 60 includes a memory, delays, amplifiers, transistors, phase rotators, and/or other devices arranged in channels. Each channel generates a transmit waveform for a given element 54. The channels are associated with specific elements 54. Alternatively, a multiplexer allows channels to connect with different elements 54 at different times.

FIG. 4 shows one embodiment of the transmit beamformer 60. The channels of the transmit beamformer 60 include transmitters 70. High efficiency transmitters with output transistor stages driven to saturation may be used. For example, each transmitter 70 is a field-effect transistor, but other waveform generators may be used. The source of the transmitters 70 connect with a high voltage (e.g., 50-120 volt) rail. Multiple rail voltages may be provided for amplitude apodization. By turning the transmitters 70 on and off, square waves are generated for the corresponding elements 54.

In alternative embodiments, sinusoidal waves are generated. The transmitters 70 of typical high power focused ultrasound systems use linear transmitters with impedance matching circuit elements, which create a sinusoidal drive waveform with the objective of minimum harmonic distortion. This approach has an upper limit of 50% for the electrical efficiency of the transmitter stages.

The transmit beamformer 60 causes the waveforms for different elements to be generated in synchronization. By introducing relative delays and/or phase shifts, a transmit beam focused at one or more locations may be generated. The delays and/or phase shifts account for the different distances from the elements 54 to a treatment location. Any steering may be used and is implemented by the transmit beamformer 60. Apodization may or may not be provided, such as amplifying or generating waveforms with different amplitude for different channels.

The transmit beamformer 60 causes the transducer array 18 to form a therapeutic beam of acoustic energy. Any dose or power may be output. For example, acoustic power greater than 100 Watts continuous wave power is generated.

The controller 66 is a transmit beamformer controller. A processor, application specific integrated circuit, analog circuit, digital circuit, memory, combinations thereof, or other device may be used. The controller 66 receives high level commands through the communications interface 68 and processes the commands to configure the transmit beamformer 60. For example, a focal location is received. The controller 66 determines the delays and/or phase shifts for steering to the focal location. The delays and/or phase shifts may be loaded from memory or calculated. As another example, the frequency and/or amplitude is set by the controller 66. In another embodiment, the transmit beamformer 60 determines the delays and/or phase shifts so that the controller 66 controls the transmit beamformer 60 over fewer wires (e.g., single wire or high speed serial bus).

In yet another example, a mode control signal is used to configure the transmit beamformer 60. The controller 66 selects a frequency, power, aperture (number and which elements 54), waveform amplitude, duty cycle, and/or other characteristic based on the mode. The mode may be for a test or sample therapeutic transmission. The effects (e.g., displacement or temperature change) of the tissue in response to the sample may be detected by the MR system 14. The focal location or other characteristics may be altered based on the feedback from the MR system 14 in order to more accurately steer for therapy. The mode may be for therapy. The duration, frequency, amplitude, power, dose, aperture, location, sequence of locations, duty cycle, or combinations thereof are set for therapy. The mode may be for elasticity imaging, such as setting the transmit beamformer 60 to cause tissue displacement.

The controller 66 may configure the transmit beamformer 60 for response to a trigger input. The therapy may operate in conjunction with monitoring by the MR system 14. The controller 66 causes the transmit beamformer 60 to generate waveforms for therapy when triggered by the MR system 14 or in synchronization with the MR system 14. The scanning or imaging by the MR system 14 may be interleaved with the therapy, so the triggering may be repeated.

By collocating the drive amplifiers (e.g., transmitters 70) and the multi-dimensional array 18 of elements 54, there is no bundle of coax cables. Instead, control signals are received by the controller 66. The controller 66 communicates over one or more traces or signal lines within the housing 58 to the adjacent (e.g., 0.1-10 cm away) transmit beamformer 60. Without the need to manage a large number of coax cables or other impedance controlled methods of interconnection, the array 18 may be finely divided (e.g., hundreds or thousands of elements) to steer the beam without a need for robotic aiming or other supplemental mechanical motion control.

By positioning the transmit beamformer 60 adjacent to the elements 54, the electrical impedance mismatch associated with feet of coaxial cabling may be less. A mismatch may still occur due to the capacitance of the elements 54. The elements 54 are formed, in part, from spaced apart electrodes, such as a signal electrode spaced from the ground foil 50 by PZT.

The matching layer 52 may be used to off-set the capacitance of the elements 54. The matching layer 52 is an epoxy, silicone, or other material. The matching layer 52 may or may not include particles of a desired density, such as conductive tungsten particles. The matching layer 52 material is chosen to have a density in-between that of the elements 54 and the patient. The density is selected to gradually transition the acoustic impedance, allowing for better transmission efficiency of the acoustic energy. To avoid reflections or attenuation, the matching layer 52 may typically be about ¼ an ultrasound wavelength in thickness. The thickness and material may be based on a desired band shape or bandwidth of operation. More than one matching layer may be used for a gradual acoustic impedance transition.

The matching layer 52 may also affect the electrical impedance from the element 54 to the transmitters 70 of the transmit beamformer 60. Since long cabling is not used, the thickness of the matching layer 52 may off-set a capacitance of the elements 54. The off-set provides a phase angle of electrical impedance within about 10 degrees of zero. Lesser or greater tolerance may be used. The transmitters 70 may be electrically matched to the acoustic array elements 54 using the mechanical matching rather than electrical inductive matching. The acoustic matching layer 54 is tuned with the objective of a zero degree phase angle in the electrical impedance of each element 54 at the operating frequency. This maximizes the power efficiency of the electrical transmitters 70. Since the bandwidth of the acoustic energy may be more limited in therapy than for imaging, the thickness of the matching layer 54 is tuned for electrical matching instead of or in addition to an acoustic objective. In one embodiment, the matching layer 52 is thinner than ¼ an ultrasound wavelength. For example, a material (e.g., graphite) with an acoustic impedance of around 6 MRayls with a thickness of about 1/7^{th} of a wavelength. This may reduce bandwidth, but also may avoid a low power factor (e.g., improve power transfer).

The connection between the transmitters 70 of the transmit beamformer 60 and the elements 54 may be free of inductors. Separate inductors for electrical impedance matching may be avoided by setting the matching layer 52 based on electrical impedance. This may result in a greater efficiency (i.e., (acoustic power delivered/electrical mains power consumed by the entire system)×100), such as greater than 20%.

Passive or active cooling may be provided. For passive cooling, thermally conductive materials may transfer heat away from an emitting face of the transducer array 18 and/or from the transmit beamformer 60.

In one embodiment, one or more fluid channels 62, 63 are provided. The fluid channels 63 are between elements 54 and/or transmit beamformers 60 (or transmitters 70). The fluid channels 63 allow fluid to flow by or be in thermal contact with elements 54, such as on the sides of elements 54. The fluid channels 63 may be above or below the elements 54, such as routing fluid through the backing.

The fluid channels 63 have any spacing, such as being by every element. For example, every two, four, or more elements 54 are spaced apart by the fluid channel 63. The fluid channels 63 interconnect. Alternatively, each fluid channel 63 is a closed loop. The fluid channels 63 extend in any direction, such as being in parallel along one dimension of the array 18.

In one embodiment, the fluid channels 63 are formed by space between modules. The housings 54 of the modules provide a barrier for the fluid channels 63. The ground foil 50 provides another barrier. A membrane, plate, or other material encloses the fluid channels 63 from a bottom (i.e., spaced away from the emitting face). Where the fluid channels 63 are formed by the modules, the fluid channels 63 interconnect in a checker board pattern through the 16×16, 2×8, or other arrangement of modules. Other boundaries may be used, such as the elements 54 themselves or chips themselves. The fluid channels 63 may extend into or through the modules (housing 58).

Structural foam may be used for the mechanical support of the modules, and the housing 58 may be made with a metal foil to which the ASICs are bonded. This may allow different shaped cooling channels, for example, where the structural foam is triangular in cross-section allowing for increased cooling channel width at the back or bottom surface of the channel.

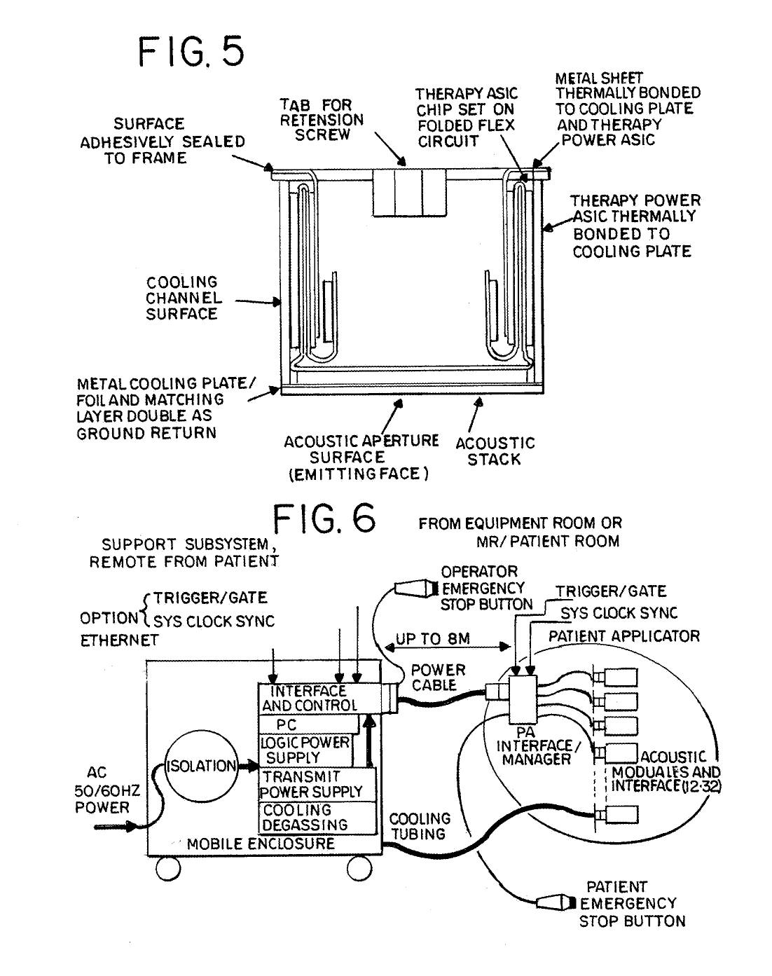

By positioning the semiconductor chips of the transmit beamformer 60, such as the semiconductor chips with the transmitters 70, adjacent to the thermally conductive housing 58, the fluid channels 63 running by the other side of the housing 58 may act as a heat sink. FIG. 5 shows an example of a module with chips adjacent the housing. Heat generated by the transmit beamformer 60 may be drawn or carried away through the 0.5 mm thick housing 58. Other thicknesses may be used. The housing 58 provides for thermal communication between the transmit beamformer 60 and the fluid channels 63. Waste heat from the electronics in the patient applicator 21 is coupled by a very short thermal path (e.g., 5 mm) to the fluid.

An alternative or additional fluid channel 62 is across the emitting face of the transducer 18. The fluid channel 62 is bounded on a bottom side by the ground foil 50 and/or matching layer 52. Where the ground foil 50 extends over all the modules or elements 54, the ground foil 50 acts as a lower barrier. Gaps connected with the fluid channels 63 may or may not be provided in the lower barrier. In one embodiment, the ground foil 50 is not continuous, so the fluid channels 63 connect with the channels 62. In another embodiment, the ground foil 50 is not continuous, but the fluid channels 63 connect with the channels 62 at edges of the applicator and are sealed separately between modules.

A membrane 64 acts as an upper barrier. The membrane 64 is flexible material with an acoustic impedance near water. For example, the membrane 64 is urethane or other flexible material. The sides of the fluid channel 62 may be an overall housing of the applicator 21 or parts of the membrane 64 extending to and bonding with the ground foil 50.

The fluid channel 62 on the emitting face may be one channel extending over the entire transducer array 18. Alternatively, extensions form the membrane 64, extensions from the ground foil 50, or inserts form two or more separate channels across the emitting face.

The fluid channel 62 may provide acoustic coupling. In addition or as an alternative to an acoustic gel on the skin of the patient, the membrane 64 is placed against the patient. The fluid in the fluid channel 62 allows the membrane 64 to conform to the patient and acoustically couples the elements 54 to the patient.

Any fluid may be used. For example, water is used. More or less viscous fluids may be used.

Where both the fluid channels 63 between housings 58 and the fluid channel 62 above the elements 54 are provided, the channels are interconnected. For example, the fluid may flow from between the modules to the emitting face where separate ground foils 50 cover each module and do not extend between modules. Alternatively, specific fluid connections are used to control the flow. For example, the fluid passes through the fluid channel 62 by the patient before passing to the fluid channels 63 for transfer of heat away from the transmit beamformers 60. Any flow direction may be used.

The fluid channels 62, 63 may use the acoustic coupling fluid to transport the thermal energy away from the patient. Alternatively, separate fluid from that used for acoustic coupling is used for thermal control.

The pump and/or reservoir 69 are positioned away from the applicator 21, such as being part of or adjacent to the sub-system 22 or outside the Faraday cage of the MR system 14. Alternatively, the pump and/or reservoir 69 are positioned in the same room and/or in the bore (e.g., under the patient table 38). The pump 69 may be part of the applicator 21. In one embodiment, the fluid is transported through a connecting tube to a location where the contained heat may be dissipated through passive or active cooling. The fluid pump and/or reservoir 69 may be connected with the cooling for the cryomagnet, but with an interface transitioning the temperature of the fluid for the transducer 18 to be comfortable for the patient. The cooling may allow independent temperature control of patient contact surface.

In one embodiment, a fluid reservoir is used instead of or with the pump. For example, the fluid is not pumped through the channels 62, 63, but instead provides a thermal path with or without flow. A fluid reservoir with a heat capacity of 20 KJoules of thermal energy with less than 1 degree temperature rise is connected with the fluid, such as in the applicator 21. For short duration therapy pulses with a low duty factor, the acoustic coupling fluid may not need to be circulated, and the fluid acts as a sufficiently large thermal reservoir with passive thermal dissipation from the reservoir to the ambient environment at the patient. The channels 62, 63 also provide heat capacity (e.g., 192 Joules per degree Celsius).

The communications interface 68 is a circuit for transmitting and receiving high level controls from the sub-system 22 and to the controllers 66. In one embodiment, the communications interface 68 is an Ethernet interface. The communications interface 68 may route signals or data as addressed to the controllers 66. Alternatively, all data goes to all the controllers 66. The communications interface 68 connects with the transmit beamformers 60 through the controllers 66 for setting and causing therapeutic transmissions.

The communications interface 68 may include a connection to receive direct current power for the applicator 21. For example, a 100 volt DC connection is provided over a coaxial cable. 3-phase 10 KVA power may be provided. The power cable may include shielding and baluns to reduce electromagnetic interference. The communications interface 68 routes the power to the transmitters 70. Voltage dividers, regulartors, or other devices in the communications interface 68, controllers 66, or transmit beamformers 60 may condition the power for the digital signal processing.

The communications interface 68 may or may not include a valve or other control for the flow of fluid. For example, the tube holding the fluid connects with a valve on the communications interface 68.

The communications interface 68 communicates steering and other operation information received from the remote sub-system 22 to the controllers 66. The steering information may indicate one or more locations for therapy. The locations are communicated without channel specific data. For example, the location is a coordinate and not a delay profile. Alternatively, the location is provided as a delay profile to be applied to the aperture. The waveforms to be applied to the elements 54 are not provided over the connection to the communications interface 68. Characteristics of the channel waveforms may be indicated, such as apodization, duration, frequency and/or aperture.

Using the transmit beamformer 60 in the applicator 21 allows for a minimal number of external electrical connections, such as only two external electrical connections, one for DC power and the other for a simple communication link to supply high level therapeutic power deposition information. The data bandwidth requirement for this level of information is minimal and may be implemented in a number of ways, such as with a MR compatible optical communication. By collocating computation resources, phase and power apodization calculations may be done locally, eliminating the need for a high speed, high bandwidth communication link to an external computational engine. System control may be located in the patient applicator 21, requiring only an external monitor and keyboard, or external interface for high level commands regarding the therapeutic ultrasound energy deposition.

The communication interface 68 may include separate inputs or uses the control input for trigger and/or mode inputs. Trigger and mode control connections may be used in the external interface. Other connections may be provided using the same input or a different port. For example, an emergency stop is provided. The patient and/or operator may cause a power disconnection and/or turn off the transmitters 70.

FIG. 6 shows an example system for therapeutic ultrasound in an MR environment. A cart is the sub-system 22 and includes power and cooling for remote control and operation of the applicator 21. The cart may be moved to connect with applicators 21 at different MR systems 14. The cart may be connected to the MR system 14 for triggering or synchronization. The therapy may be triggered or synchronized with imaging. For use within the Faraday cage, the cart may include isolation for any power source. The power may be a pluggable cord-based AC main power circuit.

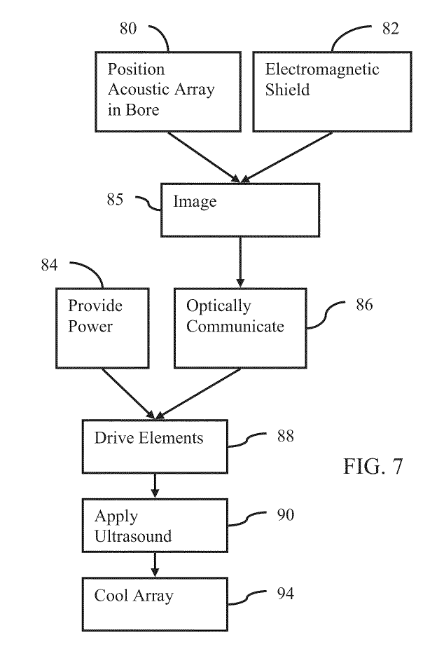

FIG. 7 shows one embodiment of a method for therapeutic ultrasound in use with magnetic resonance. The method is implemented with the transducer 18, applicator 21 of FIG. 3, system of FIG. 1, system of FIG. 2, system of FIG. 6, or different transducer, applicator, and/or system. The acts are performed in the order shown or a different order. For example, acts 85 and 86 occur in parallel. As another example, act 84 may be performed prior to acts 86, 85, 80, and 82. In yet another example, act 85 is interleaved with acts 88, 90 and 94.

Additional, different or fewer acts may be provided. For example, act 94 is not performed. Acts 80, 82, and 84 are performed for setting up the therapy in the MR environment. These acts may not be specifically performed at the time of imaging in act 85 and the application of therapy in acts 88, 90, and 94.

In act 80, an acoustic array of elements is positioned within a bore of a magnetic resonance system. The bore is a region of the MR system for imaging a patient. The array is positioned on the patient bed or on the patient. When the patient is moved into the bore for MR imaging, the array is also moved within or is within the bore.

The array may be sized and shaped to fit an indentation in the patient bed or to otherwise attached to the patient bed. Using a snap fit, clamp, bolt, clips, or other attachment, the array is connected with the patient bed.

The array is positioned as an integrated applicator. The applicator has a housing. The housing includes the array and corresponding transmitters. Controllers and/or beamformers may be enclosed by the housing. By positioning the array, the transmitters and transmit beamformer are also positioned in the bore of the MR system.

In act 82, the acoustic array and associated electronics (e.g., transmitters, beamformer, communications interface, and/or controller) are shielded. To avoid adverse imaging effects for MR imaging, the ultrasound components in the main magnetic field or bore are shielded.

Any electromagnetic shielding may be used. For example, a grounded, conductive housing surrounds the components, other than for input or output cables. The input and output cables may be shielded and connected with baluns. The housing may be compartmentalized to avoid electromagnetic interference in feedback or along signal paths. Grounding planes in printed circuit boards or at other locations may be included within the housing. The housing may have flanges or extensions that contact grounded strips on circuit boards. Filtering may be used to reduce feedback or resonation. Other shielding may be provided.

In act 84, power is provided to the drivers of the applicator. The power is provided when the MR system is turned on or configured for imaging. Alternatively, a separate power control is used. The power is cycled on and off in one embodiment. For example, the power is off whenever MR imaging is occurring. During breaks in the MR imaging sequence, the power to the applicator is turned on and therapeutic ultrasound may be generated.

The power is provided over a cable. To avoid interference, the power may be direct current. The direct current may provide a voltage for use by pulsers to generate ultrasound waveforms. Alternatively, alternating current is provided.

In act 85, the patient is imaged. Using the MR system, a sequence of radio frequency pulses in controlled magnetic fields is used to generate a response from selected molecules. Any MR sequence may be used. The response is used to generate an image. The image represents a point, line, plane, or volume (e.g., multiple planes) of the patient.

The imaging is used to locate a tumor or other region for treatment. The user and/or a processor identify the location of the treatment region. Using a coordinate transform, the location relative to the acoustic array is determined.

The imaging may alternatively or additionally be performed during or after application of therapeutic ultrasound. Using interleaving or simultaneous treatment and imaging, the progress of treatment and/or the continued accuracy of aiming the treatment at the desired location is monitored by imaging.

In act 86, control signals are communicated to the applicator, such as to a controller in the applicator. The control signals are from a user interface or other control remote from the applicator. Any type of control signals may be sent, such as location, frequency, duration, aperture, amplitude, dose, pulse repetition frequency, duty cycle, or other characteristic of the ultrasound treatment. For example, steering information is sent. The mode of operation may be sent. A trigger to activate the application of therapy may be sent.

In one embodiment, the communication is optical. Light signals are sent over a fiber optic cable. Light may not interfere with the MR imaging. Alternatively, electrical signals are sent in digital or analog form.

In act 88, the elements of the array are driven. Electrical waveforms are applied to the elements. By placing an alternating electrical waveform on one electrode and ground on an opposing electrode, a vibration is created in piezoelectric material or a capacitive membrane. The vibrations cause an acoustic wavefront to propagate from the element. By timing the wavefronts for the different elements, an acoustic beam with a point, line, area, or region focus is generated.

The electrical waveforms are generated by transmitters in the applicator and/or in the bore of the MR system. The transmitters operate in response to delays and/or phasing from a transmit beamformer. Apodization control may also be used.

The electrical waveforms for any given therapy beam may be triggered. For interleaving, the generation of therapy beams is controlled to avoid interference with MR imaging. The trigger may additionally or alternatively be for controlling when all desired arrangements have been made and the patient is ready for treatment.

In act 90, therapeutic ultrasound is applied to the patient. In response to the electrical waveforms, the therapy beam is generated. The beam is focused at a region within the patient while the patient is within the bore of the MR system.

Any level of therapy may be applied. For example, an acoustic power greater than 100 watts is transmitted from the acoustic array. Since the acoustic array has a large number of elements (e.g., at least 1,600 elements), both steering and culmination of greater powers may be generated for the therapy beam. Steering control information may indicate focal location over a range of angles, such as within a 90 degree arc or cone relative to the array. Having many elements may allow use of aperture control to shift the aperture and beam origin to different locations on the array.

In act 94, the transmitters and array are cooled. The cooling may be passive or active. Using thermally conductive material, thermal energy may be transferred or drawn away from the patient and the applicator.

In one embodiment, a fluid is used to cool. The fluid thermally conducts. A reservoir may be used to distribute or dissipate the heat. A pump may be used to transport the fluid. The fluid is heated by the applicator. The heated fluid is replaced with cooler or cooled fluid. The heated fluid is transported through a hose or other channel away from the applicator. The fluid may be cooled by refrigeration and/or radiators (e.g., fins).

The fluid may be channeled between elements of the acoustic array and/or transmit beamformers. For example, the fluid is pumped between modules of sub-arrays. The fluid may be channeled between the patient and the acoustic array. For example, the fluid is used for acoustic coupling of the array to the patient as well as cooling.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for therapeutic ultrasound in use with magnetic resonance, the system comprising:
a transducer array comprising a multi-dimensional array of elements;
a transmit beamformer connected with the transducer array;
a communications interface connected with the transmit beamformer; and
a housing electromagnetically shielding and enclosing the transducer array, the transmit beamformer and the communications interface;
wherein the transducer array, the transmit beamformer and communications interface are operable in a bore of a magnetic resonance imaging system.

2. The system of claim 1 further comprising:
a patient table in the bore;
wherein the housing connects with the patient table.

3. The system of claim 1 wherein the communications interface is configured to receive direct current power and communicate steering and operation information, the steering information indicating a location for therapy and being free of signals for the elements.

4. The system of claim 1 wherein the housing is free of a receive beamformer.

5. The system of claim 1 wherein the transmit beamformer comprises a controller and transmitters.

6. The system of claim 1 wherein the communications interface comprises a trigger input, the transmit beamformer configured to operate in response to a signal of the trigger input.

7. The system of claim 1 wherein the communications interface comprises a mode input, the transmit beamformer configured to operate based on a signal of the mode input.

8. The system of claim 1 wherein the multi-dimensional array comprises at least 1,600 elements.

9. The system of claim 1 further comprising a matching layer adjacent to the transducer array, a thickness of the matching layer off-setting a capacitance of the elements such that a phase angle of electrical impedance is within about 10 degrees of zero, connections between the transmit beamformer and the elements being free of any matching inductors.

10. The system of claim 1 wherein the transmit beamformer is configured to cause the transducer array to generate acoustic power greater than 100 Watts.

11. The system of claim 1 wherein the transducer array comprises fluid channels within the transducer;
further comprising:
a pump operable to pump fluid through the fluid channels.

12. The system of claim 11 further comprising a fluid channel across an emitting face of the transducer, the fluid comprising an acoustic coupling fluid, the fluid channel across the emitting face in fluid connection with the fluid channels within the transducer.

13. The system of claim 11 further comprising a fluid reservoir with a heat capacity of 20 KJoules of thermal energy with less than 1 degree temperature rise.

14. The system of claim 11 wherein the transducer array and transmit beamformer comprise modules with an metallic housing, a semiconductor chip of the transmit beamformer positioned against the metallic housing, a ground foil sealing an end of the metallic housing, a sub-array of the elements positioned against the ground foil, and the fluid channels being between the modules.

15. A method for therapeutic ultrasound in use with magnetic resonance, the method comprising:
positioning an acoustic array of elements distributed multi-dimensionally within a bore of a magnetic resonance system;
driving the elements with transmitters within the bore;
applying therapeutic ultrasound to a patient within the bore in response to the driving; and
imaging the patient with the magnetic resonance system.

16. The method of claim 15 wherein positioning comprises placing a housing enclosing the acoustic array on a patient bed of the magnetic resonance system, the housing also enclosing the transmitters;
further comprising:
electromagnetically shielding the acoustic array and the transmitters with the housing.

17. The method of claim 15 wherein applying comprises applying an acoustic power greater than 100 watts from the acoustic array, the acoustic array comprising at least 1,600 elements.

18. The method of claim 15 further comprising:
providing power to the drivers with a direct current over a cable; and
optically communicating trigger and steering control information to a controller housed with the transmitters;
wherein driving comprises driving in response to the trigger control information; and
wherein applying comprises applying at a steering direction responsive to the steering control information.

19. The method of claim 15 further comprising:
cooling the transmitters with a fluid.

20. The method of claim 19 wherein cooling comprises transporting fluid between modules, each of the modules including elements and transmitters.
